(19) United States
(12) Patent Application Publication
(10) Pub. No.: US 2002/0102611 A1
(43) Pub. Date: Aug. 1, 2002

(54) Combinatorial chemistry encore technique

(76) Inventor: Viktor Krchnak, Tucson, AZ (US)

Correspondence Address: Gavin J. Milczarek-Desai, Durando Birdwell & Janke PLC, 2929 E. Broadway Blvd., Tucson, AZ, 85716, US

(21) Appl. No.: 09/954,318

(22) Filed: Sep. 17, 2001

Related U.S. Application Data
(60) Provisional application No. 60/264,485, filed on Jan. 26, 2001.

(51) Int. Cl. G01N 33/53; C12M 1/34

(52) U.S. Cl. 435/7.1; 435/287.1

(57) ABSTRACT

The invention relates to an inventive method for tracking the identity of chemicals or compounds attached to individual solid phase particles during combinatorial synthesis. A particularly preferred coding method, termed “ENCORE” for short, involves Encoding by a Necklace, Color, and Reaction vessel. The Encore technique preferably combines three different coding methods: sequential position on a necklace for the first combinatorial step, color coding of individual necklaces for the second combinatorial step, and reaction vessel coding as the indication of the identity of the last building block. Two novel techniques for integrated assembly of necklaces also are described, as are novel dedicated tools for manual or automatic necklace assembly and manipulation.

### STATEMENT OF RELATED APPLICATION

This filing is based upon U.S. Provisional Patent Application No. 60/264,485, filed on Jan. 26, 2001.

### BACKGROUND OF THE INVENTION

1. Field of the Invention

This invention relates in general to the field of combinatorial chemistry and in particular to a method and apparatus for enhanced combinatorial synthesis of chemical compound libraries.

2. Description of the Related Art

The split and mix concept for combinatorial synthesis of compounds on solid phase particles, introduced by Arpad Furka and later independently used by Kit Lam and Richard Houghten is an efficient method for production of a chemical library. For further background, see Furka, A., Sebestyen, F., Asgedom, M., Dibo, G. 10^{th} International Symposium on Medicinal Chemistry (Budapest) 1988, p. 288; Furka, A., Sebestyen, F., Asgedom, M., Dibo, G. 14^{th} International Congress of Biochemistry (Prague) 1988, p. 47; Furka, A., Sebestyen, F., Asgedom, M., Dibo, G. Int. J. Pept. Protein Res. 1991, 37, 487-493); Lam, K. S., Salmon, S. E., Hersh, E. M., Hruby, V. J., Kazmierski, W. M., Knapp, R. J. Nature 1991, 354, 82-84; and Houghten, R. A., Pinilla, C., Blondelle, S. E., Appel, J. R., Dooley, C. T., Cuervo, J. H. Nature 1991, 354, 84-86. This concept requires minimum instrumentation and reduces the number of reaction vessels handled at any time to the number of building blocks used at a given combinatorial step.

However, in order to know the structural information of a compound on any given solid phase particle, there is a need to track the chemical history of particles during combinatorial synthesis. Different methods of coding exist in order to identify the specific structure of a compound associated with a given particle at a particular time during the chemical synthesis operation. Such methods have included (1) chemical coding on resin beads (See Kerr, J. M., Banville, S. C., Zuckermann, R. N., J. Am. Chem. Soc. 1993, 115, 2529-2531; Nikolaev, V., Stierandova, A., Krchnak, V., Seligmann, B., Lam, K. S., Salmon, S. E., Lebl, M. Peptide Res. 1993, 6, 161-170; Ohlmeyer, M. H. J., Swanson, R. N., Dillard, L. W., Reader, J. C., Asouline, G., Kobayashi, R., Wigler, M., Still, W. C. Proc. Natl. Acad. Sci. USA 1993, 90, 10922-10926), (2) radio-frequency tagging (See Moran, E. J., Sarshar, S., Cargill, J. F., Shahbaz, M. M., Lio, A., Mjalli, A. M. M., Armstrong, R. W. J., Am. Chem. Soc. 1995, 117, 10787-10788; Nicolaou, K. C., Xiao, X. Y., Parandoosh, Z., Senyei, A., Nova, M. P. Angew, Chem., Int. Ed. 1995, 34, 2289-2291), (3) color tagging (See Guiles, J. W., Lanter, C. L., Rivero, R. A. Angew. Chem. Int. Ed. 1998, 37, 926-928), and (4) necklace coding (See Smith, J., Gard, J., Cummings, W., Kaniszai, A., Krchnak, V. J. Comb. Chem. 1999, 1, 368-370; Furka, A., Christensen, J. W., Healy, E., Tanner, H. R., Saneii, H. J. Comb. Chem. 2000, 2, 220-223; Furka, A. Combinatorial Chemistry & High Throughput Screening 2000, 3, 197-210).

One popular coding method, the so-called necklace coding concept, organizes individual particles into a linear sequence of particles (resembling a necklace) such that the identity of any particle is determined by its position in the linear sequence. Practically any particle known to be useful in chemical synthesis applications may be used.

To accommodate micromolar quantities of material per single particle, Mimotopes has developed a product known as a SynPhase Lantern™ (Mimotopes, Clayton, Victoria, Australia; ordering information available through the mimotopes.com website). Lanterns are a modular, grafted solid-phase support resembling the cylindrical shape of a lantern, with a standard size of 5 mm×5 mm (two Lantern sizes currently are available). Each Lantern can be loaded with up to 15 or 35 umol per Lantern, depending on which size is used.

However, the “necklace method” is neither convenient nor practical for the creation of combinatorial chemical synthesis libraries. Moreover, in addition to the difficulties inherent in keeping track of the identity of a given particle at a given step, a second inherently unfavorable feature of the split and mix method is that the quantity of synthesized material is given by the amount obtained from one single particle (unless particles are combined, e.g. in a form of a T-bag; see Houghten, R. A. Proc. Natl. Acad. Sci. USA 1985, 82, 5131-5135).

Thus, there remains a need in the art for a method and apparatus for performing combinatorial chemical synthesis such that the structural information of a compound on any given solid-phase particle is known with ease and precision at any time during the synthesis process.

### SUMMARY OF THE INVENTION

The invention relates to a novel method for tracking the chemical history of a combinatorial synthesis operation based on a combination of three different coding techniques whereby the identify of compound attached to a solid particle at any given step in a chemical synthesis is determinable. Preferably, necklace coding is employed for the first combinatorial step, color-coding for the second step, and reaction vessel coding in the third combinatorial step. Consequently, a convenient name for this preferred method is the Encore method, Encoding by a Necklace, Color, and Reaction vessel.

In contrast to the directed sorting method, which uses a radio-frequency tag, the Encore method does not require redistribution of solid phase particles after each combinatorial step. In fact, the particles are handled on an individual basis only once during the synthesis, when the particles are organized into a linear sequence to form the necklace.

The Encore method represents a very simple and cost effective approach for combinatorial solid phase synthesis on modular support. The manual and automated techniques for necklace assembly can be advantageously used for various particles and containers including, but not limited to, Lanterns and NanoKans (Ordering information can be found at the irori.com website).

Furthermore, the necklace assembly process and apparatus used in conjunction with the Encore method can be employed any time a linear sequence of particles is required, including, but not limited to, general necklace coding (See Smith, J., Gard, J., Cummings, W., Kaniszai, A., Krchnak, V. J. Comb. Chem. 1999, 1, 368-370) and the spatially addressable split procedure (See Furka, A., Christensen, J. W., Healy, E., Tanner, H. R., Saneii, H. J. Comb. Chem. 2000, 2, 220-223; Furka, A. Combinatorial Chemistry @ High Throughput Screening 2000, 3, 197-210).

The Encore method of combinatorial chemical synthesis preferably includes the steps of (1) loading x number of reaction vessels with a solid support (e.g. a Lantern) and all chemical components such that the first combinatorial synthesis step is performed; (2) constructing identical necklaces with the Lanterns such that each necklace contains the same number of Lanterns (one Lantern from each reaction vessel, with each necklace distinguished by a color-coding tag); (3)placing the necklaces into individual reaction vessels, with each vessel holding necklaces of the same color; (4) performing the second combinatorial step; (5) re-arranging the necklaces before the third combinatorial step such that each reaction vessel contains a necklace with a different color tag; (6) Performing the third combinatorial step; (7) placing the individual Lanterns into a 96-well plate, with one Lantern per well; and (8) cleaving the target compounds.

Two novel tools for assembling particles into a linear sequence (i.e. on the necklace) are also included in the invention. The manual necklace assembly process is accomplished by a Lantern delivery tool and the Lantern receiving tool. The automated necklace assembly process is performed on a dedicated XYZ robotic workstation using a special tool for Lantern transfer called a Lapis tool.

A primary object of the invention is to provide an improved combinatorial chemical synthesis method.

Another object of the invention is to provide a combinatorial chemical synthesis system that keeps track of the chemical history and identity of each individual reaction product at any given time.

Yet another object of the invention is to provide an improved combinatorial chemical library synthesis method.

A further object of the invention is to provide novel and improved apparatuses for combinatorial chemical synthesis method.

Various other purposes and advantages of the invention will become clear from its description in the specification that follows and from the novel features particularly pointed out in the appended claims. Therefore, to the accomplishment of the objectives described above, this invention consists of the features hereinafter illustrated in the drawings, fully described in the detailed description of the preferred embodiment and particularly pointed out in the claims. However, such drawings and description disclose but one of the various ways in which the invention may be practiced.

### BRIEF DESCRIPTION OF THE DRAWINGS

FIG. 1 schematically depicts the preferred logistics of the Encore technique. The large box represents individual reaction vessels. Sequential numbers 1, 2, and 3 indicate individual building blocks for any combinatorial step. Consequently, a target compound labeled, e.g. 322, was made using building blocks number 3, 2, and 2 in the 1^{st}, 2^{nd}, and 3^{rd} combinatorial steps, respectively.

FIG. 2 schematically depicts a Lantern rack used in conjunction with the invention.

FIG. 3 shows a magnified, cross-sectional view of an individual well of the Lantern rack of FIG. 2. Within the well is a Lantern.

FIG. 4 is a front view of the preferred Lapis tool holding five Lanterns.

FIG. 5 shows in cross-section one shaft of a Lantern receiving tool. The shaft is filled with Lanterns attached to the Lapis tool of FIG. 4.

FIG. 6 shows a top view of the preferred Lantern releasing tool.

FIG. 7 shows the Lapis tool of FIG. 4 picking up a Lantern from a well of the Lantern rack of FIG. 2.

FIG. 8A-8C schematically depict the preferred Lantern delivery tool from a side (A & B) and top (C) view.

FIG. 9 schematically shows the preferred Lantern receiving tool for manual necklace assembly.

FIGS. 10A and 10B depict a manual necklace assembly. The Lantern delivery tool filled with Lanterns was placed on the top of the Lantern receiving tool (A). The bottom was then removed and the Lanterns dropped into the shafts of the Lantern receiving tool (B)

FIG. 11 schematically depicts the preferred Lantern plating tool used to transfer Lanterns harboring finished synthesis products to welled plates.

### DETAILED DESCRIPTION OF THE INVENTION

Description of the Preferred Encore Concept

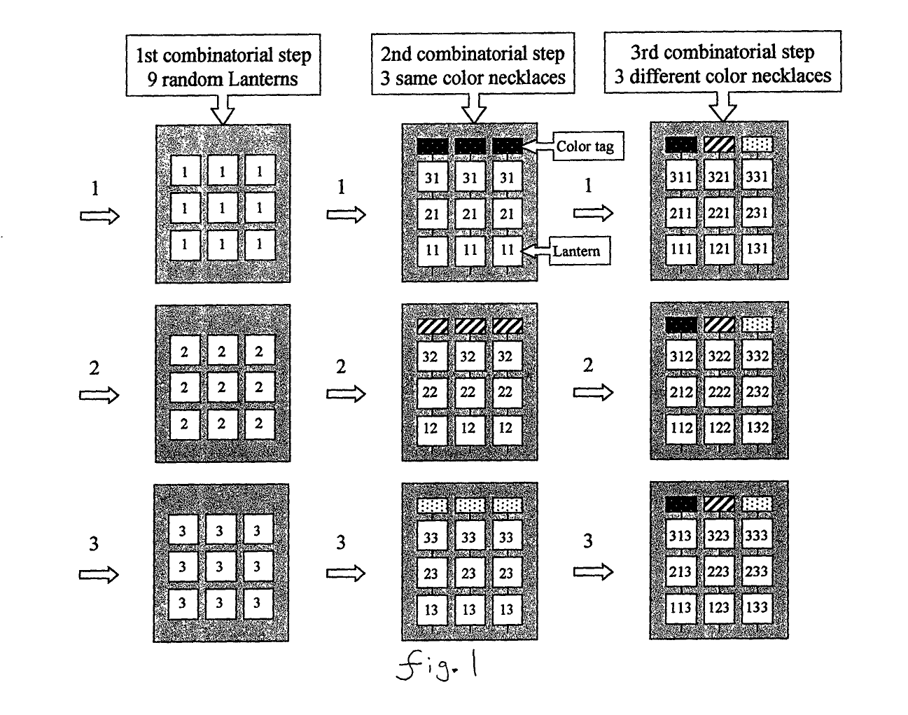

The logistics of the Encore concept is best illustrated by an example. In this example, the synthesis of a small combinatorial array of 27 compounds using 3 building blocks in each of 3 combinatorial steps is carried out. Accordingly, the preferred inventive method would consist of the following steps (see FIG. 1):

(i) Three reaction vessels are loaded with 9 Lanterns each and the first combinatorial step is performed.

(i) Nine identical necklaces are made before the second combinatorial step. Each necklace contains 3 Lanterns, one Lantern from each reaction vessel. The necklaces are distinguished by a color-coding tag. Three different colors are used (3 necklaces for each color).

(ii) Three reaction vessels are charged with necklaces, with each vessel having three necklaces of the same color.

(iii) The second combinatorial step is performed.

(iv) The necklaces are re-arranged before the third combinatorial step such that each reaction vessel contains three necklaces with a different color tag.

(v) The third combinatorial step is performed.

(vi) The individual Lanterns are placed into a 96-well plate, with one Lantern per well (not shown).

(vii) The target compounds are cleaved and isolated (not shown).

It should be understood that any number of grafted modular solid support particles could be used in place of the preferred Lantern, such as, but not limited to, resin plugs, T-bags, Microkans, Nanokans, derivatized membrane, and the like.

In order to arrange Lanterns for sizable libraries (more than 1,000 compounds), the invention includes an apparatus that facilitates automated necklace formation. The instrument is composed from the following components.

Description of the Tools for Automated Encore

Lantern Rack

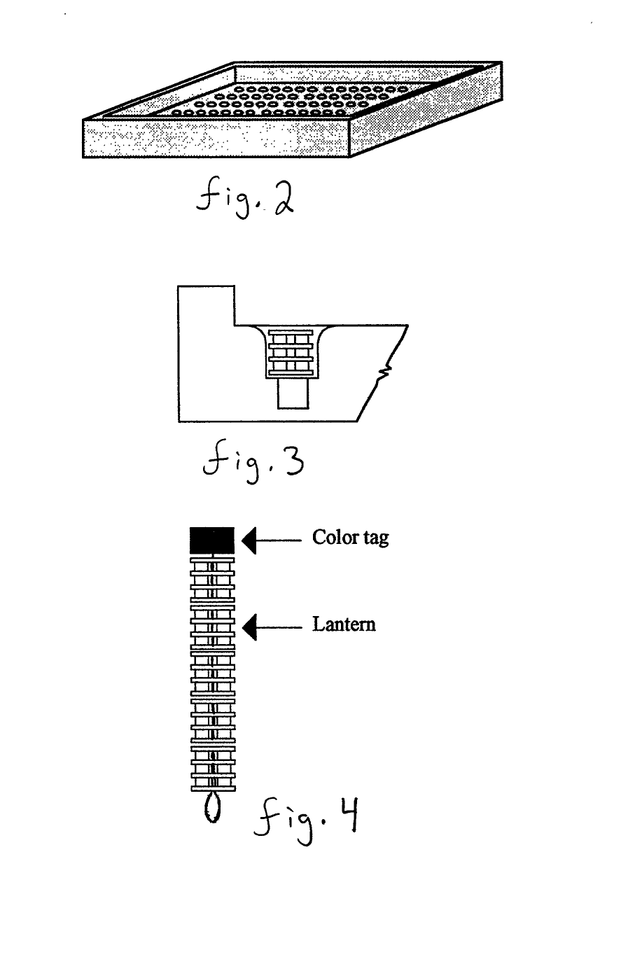

The Lantern rack (FIG. 2) is a plastic manifold containing densely arrayed cavities that approximate the size of a Lantern, with a barrier around the perimeter of the manifold. The entrance to the cavities has a specific shape to allow for smooth dropping of Lanterns into the cavities (FIG. 3). The Lantern rack serves the purpose of positioning Lanterns in a defined array. The Lantern rack is placed on the deck of a XYZ robotic workstation to begin the automated Encore process.

Lapis Tool

The Lapis tool is a Lantern picking and stringing tool (FIG. 4). Preferably, the tool is an elongated rod that is made of stainless steel or a plastic material, and can be manufactured to any reasonable length so as to accommodate a required number of Lanterns. The enlargement or loop at the tip of the tool prevents Lanterns from disengaging. The enlargement is only marginally greater than the opening in a Lantern, allowing for smooth disengagement of a Lantern from the Lapis tool by applying a gentle force.

The Lapis tool has two distinct applications. First, during the necklace assembly, the Lapis tool allows Lanterns to be picked, stringed, and carried to another location. To accomplish this, the Lapis tool is mounted on a movable arm of an XYZ robotic workstation for automated necklace assembly. Second, the Lapis tool, labeled with a color tag, is used to pick Lanterns from the Lantern receiving tool (described below). The assembled Lanterns on the tagged Lapis tools are transferred into a reaction vessel and directly used for chemical transformations.

Automated Encore Lantern Receiving Tool

The Lantern receiving tool (FIG. 5) is a plastic cube-shape block with long vertical cavities or shafts. The diameter of the shaft is marginally greater than the Lantern diameter, and the length of the shaft is designed to accommodate all stringed Lanterns for any given necklace. Preferably, the entrance into the shaft is enlarged for a reliable entry. Depending on the chemical components of the reaction, it may be desirable to string the Lanterns on a non-reactive filament.

Thus, after the Lanterns have been strung on the Lapis tool and moved to the Lantern receiving tool, Lanterns in individual cavities may be strung on a Teflon filament to form a Lantern necklace for chemical modifications. This may be accomplished simply by securing and running the Teflon filament along the Lapis tool such that the Lanterns remain threaded on the filament when the Lapis tool is removed.

The XYZ Robotic Workstation

The XYZ robotic workstation is a commercially available module with two movable arms (liquid handling robots are built on similar XYZ robotic workstations) manufactured by a variety of companies, such as Cavro, Sunnyvale, Calif.). The Lapis tool is securely attached to the first arm such it can be moved along the z-axis (i.e. up and down).

Lantern Releasing Tool

The Lantern releasing tool (FIG. 6) is mounted on the second arm. The Lantern releasing tool is made of a stainless steel sheet metal and has a U shape opening. The opening of the releasing tool is bigger than the diameter of the Lapis tool and smaller than the diameter of the Lantern so as to allow the release of the Lanterns from the Lapis tool.

Encore Reaction Block

The Encore reaction block is a cube-shape block made of Teflon, with long vertical shafts (reaction vessels), and is similar in construction to the Lantern receiving tool. The diameter of the shaft is marginally greater than the Lantern diameter, and the length of the shaft is designed to accommodate required number of Lanterns. For chemical transformations each vessel of the Encore reaction block is closed by a cap. The standard format of the Encore reaction block is 96 shafts (reaction vessels) arrayed in eight rows and twelve columns (similar to a typical 96-well plate).

Preferably, the Encore reaction block serves as the Lantern receiving tool during the necklace assembly. The Encore reaction block is then used to perform chemical reactions on the Lanterns. The building blocks for the second and third combinatorial steps are distributed by rows and columns, respectively (i.e. one type of building block per one row or column). Washing the Lanterns between steps and after finishing the synthesis is performed using the 96-well aspirator and dispenser.

The Automated Encore Method

Automated formation of Lantern necklace is described by the combinatorial synthesis equation of n=x*y*z compounds, where x, y, and z, are numbers of building blocks in the first, second, and third combinatorial steps, respectively. For the first combinatorial step, a total number of x reaction vessels are charged with y*z Lanterns each. After the first combinatorial step the Lanterns from each individual reaction vessel are spread onto the surface of the Lantern rack and, by gently shaking the rack, the Lanterns are allowed to fall into cavities of the rack. A total number of x racks are loaded with Lanterns.

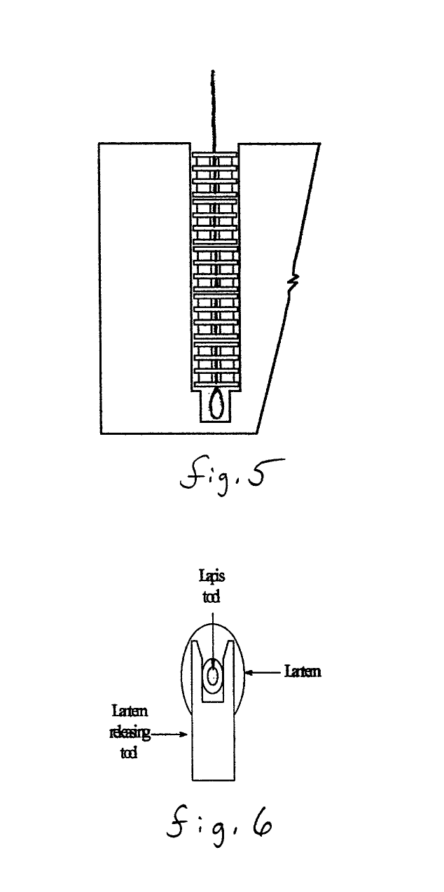

The Lantern racks, each containing Lanterns of one kind (i.e. those that received the same building block in the first combinatorial step), are placed in defined positions on the deck of the XYZ robotic workstation. The Lapis tool is then moved above the first Lantern and then slowly lowered such that it enters the opening in the Lantern (FIG. 7). The Lapis tool is then lifted above the rack. The enlargement at the end of the Lapis tool assures that the Lantern is picked and moved on the tool. The Lapis tool is then moved above the next Lantern, lowered, and the Lantern is picked. This sequence of steps is repeated x-times. As a result of this operation, the Lapis tool contains a string of x Lanterns, and the position of any Lantern defines the kind of first building block associated with that Lantern.

The Lapis tool is then moved above the Lantern receiving tool and all Lanterns are dropped into the shaft. The second arm of the robot holding the Lantern releasing tool (FIG. 6) is moved above the stringed Lanterns so that the Lapis tool enters the U shape opening. The Lapis tool is then lifted. The Lanterns remain in the shaft of the Lantern receiving tool. The Lantern releasing tool prevents Lanterns from leaving the shaft of the Lantern receiving tool.

The empty Lapis tool is then used to assemble the next sequence of Lanterns and to deliver them into the next shaft of the Lantern receiving tool. When all Lanterns are moved from the Lantern racks to the Lantern receiving tool, a total number of y*z shafts are filled with x Lanterns each one. In the next step, all Lanterns from a shaft are picked using the tagged Lapis tool. Lanterns on the tagged Lapis tools can be directly used for chemical transformations. Alternatively, Lanterns can be stringed on a Teflon rope to make the necklace. There are y*z necklaces formed, with each necklace being color-coded. A total number of y colors are used and z necklaces are color-coded with the same color. Alternative labeling of necklaces also can be used (e.g., numbering).

The next combinatorial step is performed in y reaction vessels, with each reaction vessel charged with z necklaces of the same color. For the third combinatorial step, the necklaces are placed into z reaction vessels, with each vessel charged with y necklaces of a different color.

Compounds are cleaved from the solid support after finishing the synthesis in standard 96-well plates, with one compound (i.e. one Lantern) per well. Lanterns are distributed into individual wells on the XYZ robotic workstation. Lanterns from the necklaces are transferred into shafts of the Lantern receiving tool, maintaining the order of Lanterns.

The Lantern receiving tool then is placed on the XYZ robotic workstation together with the 96-well plates. The Lapis tool is moved into the first shaft all the way to the bottom to pick all Lanterns from the shaft. The Lapis tool is then moved above the first well. The Lantern releasing tool is moved above the stack of Lanterns and moved down to release one Lantern at a time from the stack. The Lapis tool subsequently is moved above the second well, and the second Lantern is released into the well. All Lanterns from all shafts are distributed in a particular order in this way.

In an alternative arrangement of the apparatus, the XYZ robotic workstation is equipped with only one arm. Both the Lapis tool and the Lantern releasing tool are mounted onto this arm such that they can be independently moved along the z-axis. The necklace formation and other steps follow analogous steps as described for the two arms system. In another variation on the preferred apparatus, the Lantern releasing tool is attached to the Lantern receiving tool. The Lantern releasing tool is positioned the way that the entry into the shaft is not blocked. After the Lapis tool with Lanterns enters the shaft, the Lantern releasing tool is moved so that the Lapis tool penetrates the U shape opening. Thus, as the Lapis tool is lifted, the Lanterns remain in the shaft because the shaft entrance is blocked.

To distribute Lanterns from the Lantern receiving tool, one Lantern at a time is picked by the Lapis tool and moved above the well. The Lapis tool then is moved upwards, leaving the Lantern releasing tool stationary. The Lantern is released when the tip of the Lapis tool passes through the Lantern releasing tool.

The automated assembly of necklaces is not limited to the Encore technique; it can be used whenever a sequence of particles needs to be formed. For example, a sequence may be made by placing particles in a tube, or by sticking one to another, in a pre-determined sequence, whereby the position of each particle determines the identity of the chemical or compound attached to it. Accordingly, the apparatus can be programmed to create any sequence of Lanterns.

Description of the Tools for Manual Encore

Lantern Delivery Tool

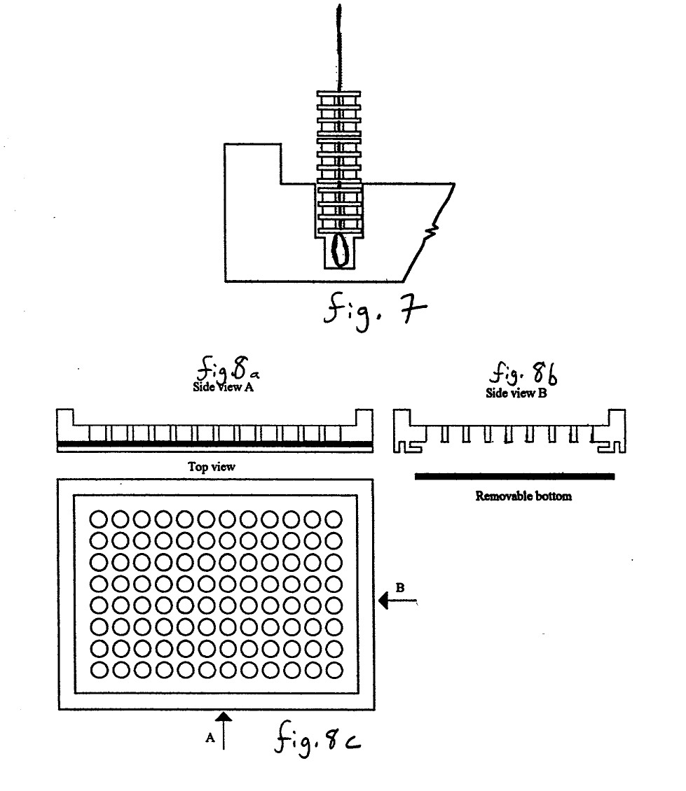

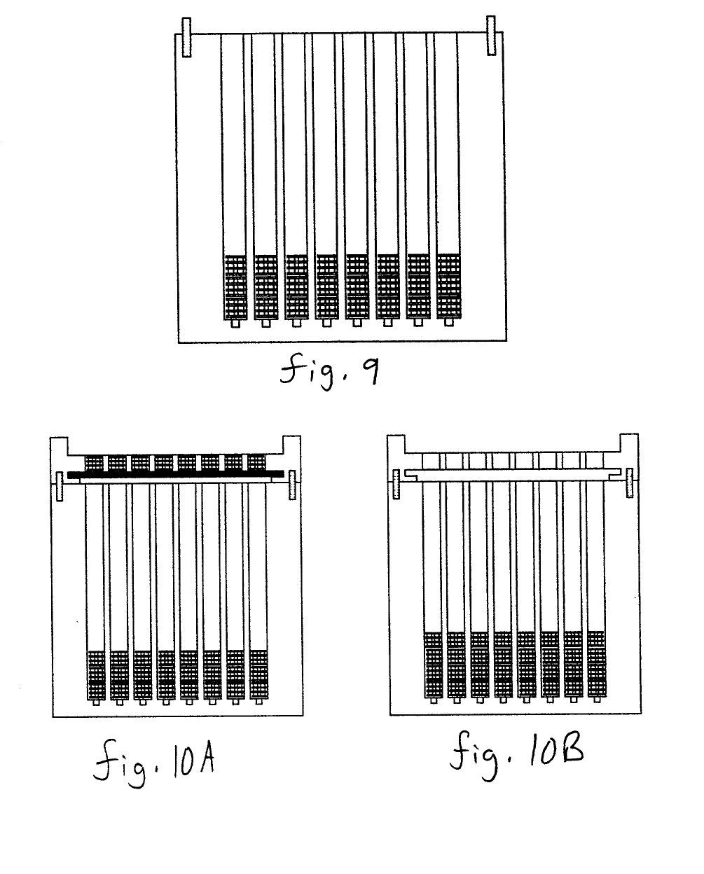

The Lantern delivery tool (FIG. 8) is a plastic manifold containing densely arrayed cavities of a Lantern size with a barrier around the perimeter of the manifold. The cavities have a specific shape to allow a smooth dropping of Lanterns into cavities. Moreover, the bottom of the tool is made of stainless steel sheet and it is removable. The Lantern delivery tool serves the purpose of positioning and distributing Lanterns in a defined array.

Lantern Receiving Tool

Similar to the tool employed for the automated Encore method, the manual Lantern receiving tool (FIG. 9) is a plastic cube-shape block with long vertical cavities. The diameter of the shaft is marginally greater than the Lantern diameter and the length of the shaft is designed to accommodate required number of Lanterns. Four pins enable exact positioning of the Lantern delivery tool on the top of the Lantern receiving tool.

Encore Reaction Block

The Encore reaction block is a cube-shape block made of Teflon, with long vertical shafts (reaction vessels), and is similar in construction to the Lantern receiving tool. The diameter of the shaft is marginally greater than the Lantern diameter, and the length of the shaft is designed to accommodate required number of Lanterns. For chemical transformations each vessel of the Encore reaction block is closed by a cap. The standard format of the Encore reaction block is 96 shafts (reaction vessels) arrayed in eight rows and twelve columns (similar to a typical 96-well plate). Preferably, the Encore reaction block serves as the Lantern receiving tool during the necklace assembly. The Encore reaction block is then used to perform chemical reactions on the Lanterns. The building blocks for the second and third combinatorial steps are distributed by rows and columns, respectively (i.e. one type of building block per one row or column). Washing the Lanterns between steps and after finishing the synthesis is performed using the 96-well aspirator and dispenser.

Description of the Manual Encore Method

Manual assembly of Lantern necklaces is described by the combinatorial synthesis equation of n=x*y*z compounds, where x, y, and z, are numbers of building blocks in the first, second and third combinatorial steps, respectively. For the first combinatorial step, a total number of x reaction vessels are charged with y*z Lanterns each. After the first combinatorial step, the Lanterns from the first reaction vessel are spread onto the surface of the Lantern delivery tool. By gently shaking the rack, the Lanterns are allowed to fall into cavities of the rack.

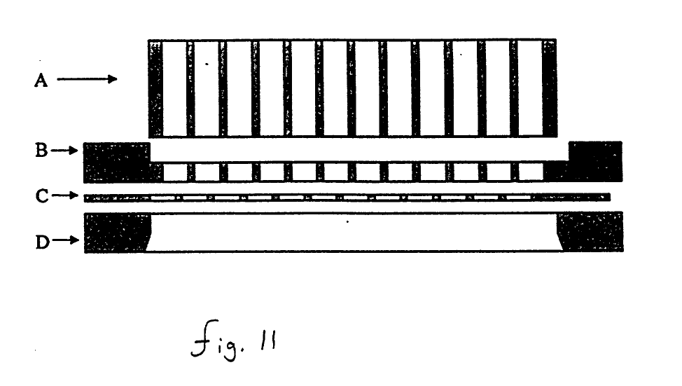

The Lantern delivery tool containing Lanterns of one kind (i.e. those that received the same building block in the first combinatorial step) is placed on the top of the Lantern receiving tool. Four dowels enable exact alignment and positioning of the tools. The movable bottom of the Lantern delivery tool is removed, and the Lanterns are dropped into the Lantern receiving tool (FIG. 10). The bottom is then returned to its original position and Lanterns from the next reaction vessel are spread on the Lantern delivery tool. When all the Lanterns are distributed from all reaction vessels into the Lantern delivering tool, and transferred to the Lantern receiving tool, a total number of y*z shafts are filled, with x Lanterns each one.

Compound Release Tool

After finishing the combinatorial synthesis, all the Lanterns are stringed on the Lapis tools. In order to release the target compounds from the Lanterns, individual Lanterns preferably are placed into a convenient integrated reaction vessel for cleavage of compound from Lanterns. The typical integrated reaction vessel is a 96-well plate. Accordingly, Lanterns are distributed in wells of a plate, one Lantern per well. The Lantern plating tool enables transfer Lanterns from Lapis tools into wells of a plate.

Lantern Plating Tool

Referring to FIG. 11, the preferred Lantern plating tool consists of four parts: (i) the Lantern receiving magazine A, (ii) the Lantern re-aligning manifold B, (iii) moveable bottom portion C, and, optionally, (iv) plate aligning manifold D. The Lantern receiving magazine A preferably is a polypropylene block with 96 wells marginally greater that a diameter of a Lantern (e.g. 5 mm) in an 8 by 12 array that hold up to 10 Lanterns in each well and preserves the sequence of Lanterns on the Lapis tool.

The Lantern re-aligning manifold B preferably is an aluminum block with 96 wells marginally greater that a diameter of a Lantern (e.g. 5 mm) in an 8 by 12 array. The height of the manifold is equal to the height of a Lantern (5 mm). The manifold B is located below the magazine A and is slideably attached such that it can be moved approximately 4.5 mm along the width of magazine A.

The bottom portion C preferably is made of a thin metal sheet (e.g. stainless steel) and has 96 holes marginally greater that a diameter of a Lantern in an 8 by 12 array. The bottom portion C is located bellow the manifold B and it is stationary with respect to the magazine A.

The optional plate aligning manifold D is located below the bottom portion C and it serves the purpose of aligning the 96-well plate to receive the lanterns.

The Lanterns are manually placed into the magazine A from the Lapis tool(s). At that time, the manifold B is aligned with the manifold A, and the lanterns are held in place by the bottom portion C. Then the manifold B is moved into alignment with the holes in the bottom portion C. Once the holes are aligned, the Lanterns are released into a 96-well receiving plate. The manifold B is then moved back to its starting position. The full 96-well receiving plate is replaced by an empty 96-well plate and the operation is repeated. In this manner, multiple plates are filled with Lanterns.

Various changes in the details, steps and components that have been described may be made by those skilled in the art within the principles and scope of the invention herein illustrated and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products.

All publications cited are hereby incorporated by reference in their entirety.

I claim:

1. A method for tracking the chemical identity of a compound connected to individual solid support particles, said compound resulting from a combinatorial solid phase synthesis of a plurality of chemicals, comprising the following steps:
a) positioning the solid support particles in a predetermined sequence after an initial combinatorial chemical modification, thereby producing a plurality of first sets of said sequence corresponding to the plurality of chemicals,
b) tracking each of said first sets such that an identity of a second combinatorial chemical modification added thereto is determinable, thereby producing a plurality of second sets of said sequence corresponding to said plurality of chemicals,
c) coding each of said second sets such that an identity of a third combinatorial modification added thereto is determinable.

2. The method of claim 1, wherein the solid support particles of step a) comprise a grafted modular solid support.

3. The method of claim 2, wherein the grafted modular solid support is a SynPhase Lantern.

4. The method of claim 1, wherein the solid support particles of step a) comprise a derivatized membrane.

5. The method of claim 1, wherein the solid support particles of step a) are selected from the group consisting of T-bags, MicroKans, NanoKans, and combinations thereof.

6. The method of claim 1, wherein the solid support particles of step a) comprise a resin plug.

7. The method of claim 1, wherein the solid support particles of step a) are sequentially positioned on a filament.

8. The method of claim 1, wherein the solid support particles of step a) are sequentially positioned within a tube.

9. The method of claim 1, wherein the solid support particles of step a) are sequentially positioned by attaching one solid support particle to another.

10. The method of claim 1, wherein the step of tracking each sequence of solid support particles is performed by labeling said sequence.

11. The method of claim 10, wherein the label comprises a color tag.

12. The method of claim 10, wherein the label comprises an alphanumeric code.

13. The method of claim 10, wherein the label comprises a uniquely shaped tag.

14. The method of claim 10, wherein the label comprises a radio frequency tag.

15. The method of claim 10, wherein the label comprises a bar code.

16. A process for tracking the identity of a compound attached to individual solid phase particles during a solid-phase combinatorial chemical synthesis, comprising:
(a) sequentially positioning a solid support means on a filament after a first step of said combinatorial chemical synthesis such that the identity of the compound is determinable;
(b) tracking individual filaments such that the identity of the compound after a second combinatorial chemical modification is determinable; and
(c) coding a reaction vessel such that the identity of the compound is determinable after a third combinatorial chemical modification.

17. The process of claim 16, wherein the solid support means of part (a) is selected from the group consisting of Synphase Lanterns, NanoKans, Microkans, T-bags, derivatized membrane, resin plugs, and combinations thereof.

18. The process of claim 16, wherein the step of tracking each sequence of solid support particles is performed by providing a label for each said sequence.

19. The process of claim 18, wherein the label comprises a color tag.

20. The process of claim 18, wherein the label comprises an alphanumeric code.

21. The process of claim 18, wherein the label comprises a uniquely shaped tag.

22. The process of claim 18, wherein the label comprises a radio frequency tag.

23. The process of claim 18, wherein the label comprises a bar code.

24. A method of combinatorial chemical synthesis of target compounds, comprising the steps of:
(1) loading a predetermined number of reaction vessels with a solid support means and all required chemical components such that a first combinatorial synthesis step is performed;
(2) positioning said solid support means in a predetermined sequence, such that each sequence has a uniquely identifying label;
(3) placing the sequences of solid support means of step (2) into individual reaction vessels, with each vessel holding sequences with the same label, and performing a second combinatorial synthesis step;
(4) re-arranging the sequences of solid support means of step (3) such that each reaction vessel contains a sequence with a different label and performing a third combinatorial synthesis step;
(5) distributing the sequences of solid support means of step (4) into harvesting plates; and (6) harvesting the target compounds.

25. An apparatus for arranging solid phase particles into a two dimensional array, comprising:
An elongated member having a first end and a second end, said second end having a deformable loop such that said solid phase particles are acquired or released from the apparatus through pressure applied along the axis of the elongated member.

26. An apparatus for dispensing solid phase particles into a receiving container with a plurality of compartments such that one particle at a time is distributed per compartment, comprising:
an upper magazine having a plurality of wells for receiving said solid phase particles; and
a lower manifold, including a plurality of wells having a capacity to hold one solid phase particle, and a moveable bottom portion including a plurality of holes; the lower manifold being in slideable attachment to the upper stationary magazine such that alignment with the wells of the upper stationary magazine allows the passage of a solid phase particle into the lower manifold; and the bottom portion being in slideable attachment to the lower manifold such that alignment with wells of the lower manifold results in dispensing of said solid phase particles into the compartments of the receiving container.
